(19) United States
(12) Patent Application Publication
(10) Pub. No.: US 2025/0129371 A1
(43) Pub. Date: Apr. 24, 2025

(54) Treatment Of Macular Degeneration With Complement Factor D (CFD) Inhibitors

(71) Applicant: Regeneron Pharmaceuticals, Inc., Tarrytown, NY (US)

(72) Inventors: Neelroop Parikshak, Tarrytown, NY (US); Eric Jorgenson, Tarrytown, NY (US); Xinyuan Zhang, Tarrytown, NY (US); Giovanni Coppola, Tarrytown, NY (US); Goncalo Abecasis, Tarrytown, NY (US); Aris Baras, Tarrytown, NY (US); Botir Sagdullaev, Tarrytown, NY (US); Ying Hu, Tarrytown, NY (US); Kevin Kanning, Tarrytown, NY (US); Aarin Jones, Tarrytown, NY (US); Huy Vu, Tarrytown, NY (US)

(21) Appl. No.: 18/911,994

(22) Filed: Oct. 10, 2024

Related U.S. Application Data
(60) Provisional application No. 63/543,536, filed on Oct. 11, 2023.

(51) Int. Cl. C12N 15/113; A61K 45/06; A61P 27/02; C12Q 1/6809; C12Q 1/6883

(52) U.S. Cl. CPC C12N 15/1137; A61K 45/06; A61P 27/02; C12Q 1/6809; C12Q 1/6883; C12N 2310/11; C12N 2310/14; C12Y 304/21046

(57) ABSTRACT

The present disclosure generally relates to the treatment of subjects having macular degeneration or at risk of developing macular degeneration by administering a Complement Factor D (CFD) inhibitor to the subject, and to methods of identifying subjects having an increased risk of developing macular degeneration.

### FIELD

The present disclosure generally relates to the treatment of subjects having macular degeneration or at risk of developing macular degeneration, by administering a Complement Factor D (CFD) inhibitor to the subject, and to methods of identifying subjects having an increased risk of developing macular degeneration.

### BACKGROUND

Macular degeneration is a serious medical condition resulting in loss of vision in the center of the visual field. “Dry” (“nonexudative”) and “wet” (“neovascular” or “exudative”) forms of macular degeneration have been recognized. Macular degeneration is often an age-related disease (hence it is often called age-related macular degeneration) that begins with early lipoproteinaceous deposits called drusen that progressively accumulate and are associated with pigmentary changes in the eye (intermediate-stage macular degeneration). Age-related macular degeneration (AMD) is the leading global cause of blindness, where pathology includes retinal accumulation of extracellular deposits and progressive loss of retinal pigment epithelium (RPE) and adjacent photoreceptors. Neovascular-AMD is successfully treated by VEGF antagonism. Intermediate-stage macular degeneration progresses to either neovascular or nonexudative macular degeneration. In neovascular macular degeneration, vision loss can be due to abnormal blood vessel growth (choroidal neovascularization). Proliferation of abnormal blood vessels in the retina is stimulated by vascular endothelial growth factor (VEGF). The new vessels are fragile and can lead to blood and protein leakage below the macula. Bleeding, leaking, and scarring from those blood vessels can eventually cause irreversible damage to the photoreceptors and rapid vision loss. In nonexudative macular degeneration, vision loss is due to primary loss of photoreceptor cells in the macula, resulting in large areas of cell loss known as “geographic atrophy.” For intermediate to late AMD (i.e., about 90% of all cases), there is no effective treatment.

Complement Factor D (CFD) (NCBI Gene ID: 1675) is encoded by an 8 kb gene located at 19p13.3 and is a member of the S1, or chymotrypsin, family of serine peptidases. CFD protein is 253 amino acids long and is a 27 kDa protein that functions as the rate-limiting enzyme in the alternative pathway of complement activation, a branch of the innate immune system that can respond to cell damage and is known to play a role in macular degeneration. Specifically, CFD catalyzes the cleavage factor B after it complexes with C3b, converting C3bB to C3bBb+Ba. C3bBb then proceeds to activate downstream pathways including membrane attack complex (MAC) formation that damages cells, and the Ba fragment can also proceed to cause inflammation by other mechanisms.

### SUMMARY

The present disclosure provides methods of treating a subject having macular degeneration, or at risk of developing macular degeneration, the methods comprising administering a CFD inhibitor to the subject, wherein the macular degeneration is an intermediate, wet, or dry form that may be aging related.

The present disclosure also provides methods of treating a subject having macular degeneration or at risk of developing macular degeneration by administering a macular degeneration therapeutic agent or macular degeneration therapy, the methods comprising: determining or having determined whether the subject has a CFD variant nucleic acid molecule, by: obtaining or having obtained a biological sample from the subject; and performing or having performed a sequence analysis on the biological sample to determine if the subject has a genotype comprising a CFD variant nucleic acid molecule; and administering or continuing to administer the macular degeneration therapeutic agent in an amount that is the same as or less than a standard dosage amount or macular degeneration therapy, and/or administering a CFD inhibitor to a subject that is CFD reference; administering or continuing to administer the macular degeneration therapeutic agent in an amount that is the same as or less than a standard dosage amount or macular degeneration therapy, and/or administering a CFD inhibitor to a subject that is heterozygous for the CFD variant nucleic acid molecule; or administering or continuing to administer the macular degeneration therapeutic agent or macular degeneration therapy in a standard dosage amount to a subject that is homozygous for the CFD variant nucleic acid molecule; wherein the presence of the CFD variant nucleic acid molecule indicates the subject has a decreased risk of developing macular degeneration.

The present disclosure also provides methods of identifying a subject having an increased risk of developing macular degeneration, the methods comprising: determining or having determined the presence or absence of a CFD variant nucleic acid molecule in a biological sample obtained from the subject; wherein: when the subject is CFD reference, then the subject has an increased risk of developing macular degeneration; and when the subject is heterozygous or homozygous for the CFD variant nucleic acid molecule, then the subject has a decreased risk of developing macular degeneration.

The present disclosure also provides macular degeneration therapeutic agents for use in the treatment or prevention of macular degeneration in a subject having a CFD variant nucleic acid molecule.

The present disclosure also provides CFD inhibitors for use in the treatment or prevention of macular degeneration in a subject that is CFD reference or is heterozygous for a CFD variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a graphical representation of analysis results for wet and dry AMD, split across different cohorts demonstrating the effect's replication across different patient cohorts.

FIG. 2 shows CFD missense variant modifies risk in individuals with high genetic risk for AMD based on genetic risk score stratification.

FIG. 3 shows CFD protective signal consistent in largest cohorts for burden mask and top variant. There is a protective effect for both wet and dry AMD.

FIG. 4 shows that the top variant, 19:860766:G:A (E69K), showed consistent protection for AMD and AMD subtypes.

FIG. 5 shows an interaction analysis in UKB for CFD protective variants and CFH Y402H risk variants.

FIG. 6 shows that CFD mRNA is detected in the neural retina and choroid by RNAscope.

FIG. 7 shows that CFD protein is found in retinal pigment epithelium cells and choroidal tissues by immunohistochemistry (IHC).

FIG. 8 shows CFD protein production process.

FIG. 9 shows a C3 Convertase assay with purified CFD.

FIG. 10 shows an assay validation.

FIG. 11 shows a dose-response of CFD WT and variants.

FIG. 12 shows kinetics of CFD WT and variants.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

Analysis of human genetics associations with AMD implicates the alternative pathway (AP) of the complement system as a driver of AMD, showing how multiple gene variants impact risk of AMD. In particular, it has been observed in accordance with the present disclosure that a rare CFD missense variant nucleic acid molecule (19:860766:G:A, E69K) (whether these variants are homozygous or heterozygous in a particular subject) is the rate-limiting enzyme for AP activation, and associates with a decreased risk of developing macular degeneration and is protective against AMD (AII AMD, OR=0.79, P=5.9×10⁻⁷). It is believed that it has not been previously established that CFD variant nucleic acid molecules that result in decreased CFD protein function or expression level have been associated with macular degeneration in humans. Therefore, subjects that are CFD reference or heterozygous for a CFD variant nucleic acid molecule may be treated with a CFD inhibitor such that macular degeneration is inhibited or prevented, the symptoms thereof are reduced or prevented, and/or development of symptoms is repressed or prevented. It is also believed that such subjects having macular degeneration may further be treated with one or more macular degeneration therapeutic agents or macular degeneration therapy that treats or inhibits macular degeneration. In addition, the present disclosure provides methods of leveraging the presence or absence of CFD variant nucleic acid molecules in subjects to identify or stratify risk is such subjects of developing macular degeneration, or to diagnose subjects as having an increased risk of developing macular degeneration.

For purposes of the present disclosure, any particular subject, such as a human, can be categorized as having one of three CFD genotypes: i) CFD reference; ii) heterozygous for a CFD variant nucleic acid molecule; or iii) homozygous for a CFD variant nucleic acid molecule. A subject is CFD reference when the subject does not have a copy of a CFD variant nucleic acid molecule. A subject is heterozygous for a CFD variant nucleic acid molecule when the subject has a single copy of a CFD variant nucleic acid molecule. A subject is homozygous for a CFD variant nucleic acid molecule when the subject has two copies of a CFD variant nucleic acid molecule.

In any of the embodiments described herein, the CFD variant nucleic acid molecule can be any nucleic acid molecule (such as, a genomic nucleic acid molecule, an mRNA molecule, or a cDNA molecule produced from an mRNA molecule) encoding a CFD variant polypeptide having a partial loss-of-function, a complete loss-of-function, a predicted partial loss-of-function, or a predicted complete loss-of-function. A subject who has a CFD polypeptide having a partial loss-of-function (or predicted partial loss-of-function) is hypomorphic for CFD. In some embodiments, the CFD variant nucleic acid molecule results in decreased or aberrant expression or activity of CFD mRNA or polypeptide. In some embodiments, the CFD variant nucleic acid molecule is associated with a reduced in vitro response to CFD ligands compared with reference CFD. In some embodiments, the CFD variant nucleic acid molecule is a splice-site variant, a stop-gain variant, a start-loss variant, a stop-loss variant, a frameshift variant, an in-frame indel variant, or a variant that encodes a truncated CFD variant polypeptide. In some embodiments, the CFD variant nucleic acid molecule is a missense variant nucleic acid molecule. In some embodiments, the CFD variant nucleic acid molecule comprises a single nucleotide polymorphism (SNP). In some embodiments, the CFD variant nucleic acid molecule comprises a variation in a coding region. In some embodiments, the CFD variant nucleic acid molecule does not comprise a variation in a non-coding region, except for a splice acceptor region (two bases before the start of any exon except the first). In some embodiments, the CFD variant nucleic acid molecule results or is predicted to result in a premature truncation of a CFD polypeptide compared to the reference CFD. In some embodiments, the CFD variant nucleic acid molecule is a variant that is predicted to be damaging to the protein function (and hence, in this case, protective to the human) by in vitro prediction algorithms such as Polyphen, SIFT, or similar algorithms. In some embodiments, the CFD variant nucleic acid molecule is a variant that causes or is predicted to cause a nonsynonymous amino acid substitution in a CFD nucleic acid molecule and whose allele frequency is less than 1/100 alleles in the population from which the subject is selected. In some embodiments, the CFD variant nucleic acid molecule is any rare missense variant (allele frequency<0.1%; or 1 in 1,000 alleles), or any splice-site, stop-gain, start-loss, stop-loss, frameshift, or in-frame indel, or other frameshift CFD variant.

In any of the embodiments described herein, the CFD variant genomic nucleic acid molecule may include one or more variations at any of the positions of human chromosome 19 (i.e., positions 859,453-867,884) using the nucleotide sequence of the CFD reference genomic nucleic acid molecule in the GRCh38/hg38 human genome assembly (see, ENSG00000197766, ENST00000327726.11, UniProt P00746; annotated in the Ensembl database (world wide web at “https://useast.ensembl.org/Homo_sapiens/Gene/Summary?g=ENSG00000197766;r=19:859453-867884”)) as a reference sequence. The sequences provided in these transcripts for the CFD genomic nucleic acid molecule are only exemplary sequences. Other sequences for the CFD genomic nucleic acid molecule are also possible.

In any of the embodiments described herein, the CFD variant nucleic acid molecule may comprise any one or more of the following genetic variations in the genomic nucleic acid molecule (referring to the chromosome: positions set forth in the GRCh38/hg38 human genome assembly): rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), OR 19:860686:C:A (Ser42Ter), or an mRNA molecule produced therefrom, or a cDNA molecule produced from the mRNA molecule. In any of the embodiments described herein, the variation that results in the Glu69Lys variant may also result in a Glu76Lys variant due to a variant transcript or variations in protein residue numbering based on pro-peptides.

For subjects that are genotyped or determined to be CFD reference, such subjects have an increased risk of developing macular degeneration. For subjects that are genotyped or determined to be either CFD reference or heterozygous for a CFD variant nucleic acid molecule, such subjects can be treated with a CFD inhibitor.

In any of the embodiments described herein, the subject in whom macular degeneration is prevented by administering a CFD inhibitor may be anyone at risk for developing macular degeneration including, but not limited to, subjects with a genetic predisposition for developing macular degeneration. In some embodiments, administering a CFD inhibitor to a subject having macular degeneration may be carried out to prevent development of another occurrence of macular degeneration in a subject who has already had macular degeneration. In any of the embodiments described herein, the methods can be used to improve macular degeneration.

In any of the embodiments described herein, the CFD predicted loss-of-function polypeptide can be any CFD polypeptide having a partial loss-of-function, a complete loss-of-function, a predicted partial loss-of-function, or a predicted complete loss-of-function.

Any one or more (i.e., any combination) of the CFD variant nucleic acid molecules described herein can be used within any of the methods described herein to determine whether a subject has an increased or decreased risk of developing macular degeneration. The combinations of particular variants can form a mask used for statistical analysis of the particular correlation of CFD and an increased or decreased risk of developing macular degeneration. In some embodiments, the mask used for statistical analysis of the particular correlation of CFD and an increased or decreased risk of developing macular degeneration can exclude any one or more of these CFD variant nucleic acid molecules described herein.

In any of the embodiments described herein, the subject can have macular degeneration (such as, for example, age-related macular degeneration; AMD). In any of the embodiments described herein, the subject can be at risk of developing macular degeneration. In any of the embodiments described herein, the macular degeneration can be atrophic (dry macular degeneration). In any of the embodiments described herein, the macular degeneration can be advanced neovascular (wet macular degeneration). In any of the embodiments described herein, the macular degeneration can be intermediate macular degeneration.

In any of the embodiments described herein, the methods can be used to treat a complication or co-morbidity of macular degeneration, or reduce the risk of developing the same.

The present disclosure provides methods of treating a subject having macular degeneration or at risk of developing macular degeneration, the methods comprising administering a CFD inhibitor to the subject.

In some embodiments, the CFD inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a CFD nucleic acid molecule. In some embodiments, the antisense RNA, siRNA, or shRNA hybridizes to a sequence within a CFD genomic nucleic acid molecule or mRNA molecule and decreases expression of the CFD polypeptide in a cell in the subject. In some embodiments, the CFD inhibitor comprises an antisense molecule that hybridizes to a CFD genomic nucleic acid molecule or mRNA molecule and decreases expression of the CFD polypeptide in a cell in the subject. In some embodiments, the CFD inhibitor comprises an siRNA that hybridizes to a CFD genomic nucleic acid molecule or mRNA molecule and decreases expression of the CFD polypeptide in a cell in the subject. In some embodiments, the CFD inhibitor comprises an shRNA that hybridizes to a CFD genomic nucleic acid molecule or mRNA molecule and decreases expression of the CFD polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, polyhistidine (i.e., 6×His, 10×His), glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (l), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, the siRNA molecules are conjugated to an antibody, or antigen-binding fragment thereof. In some embodiments, the antibody is an anti-CFD antibody. In some embodiments, the antibody may promote targeting to a specific tissue or cell type (i.e., an antibody to protein targets in adipose tissue, vasculature, retina, or choroid).

In some embodiments, a representative siRNA has the following formula:
Sense: mN*mN*/i2FN/mN/i2FN/mN/i2FN/mN/i2FN/mN/i2FN/mN/i2FN/mN/i2FN/mN/i2FN/*mN*/32FN/
Antisense: /52FN/*/i2FN/*mN/i2FN/mN/i2FN/mN/i2FN/mN/i2FN/mN/i2FN/mN/i2FN/mN/i2FN/mN/i2FN/mN*N*N
   wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 mg to about 640 mg (2.5 to 9.14 mg/kg; 92.5 to 338 mg/m²-based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art. In some embodiments, gene transfer vectors based on AAV can be used to express shRNA. In some embodiments, the AAV may be coupled to an antibody to allow cell and tissue specific targeting, such as transferrin receptor to enhance blood brain barrier crossing.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly (lactic acid) (PLA) microspheres, poly (D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the CFD inhibitor comprises a nuclease agent that induces one or more nicks or double-strand breaks at a recognition sequence(s) or a DNA-binding protein that binds to a recognition sequence within a CFD genomic nucleic acid molecule. The recognition sequence can be located within a coding region of the CFD gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the CFD gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a CFD genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of CFD nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a CFD genomic nucleic acid molecule or it can be a nickase that creates a single-strand break in a CFD genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA. In some embodiments, transduction with AAV vectors encoding CRISPR-Cas nucleases can be used.

In some embodiments, targeted genetic modifications of CFD genomic nucleic acid molecules can be generated by contacting a cell with a Cas protein and one or more gRNAs that hybridize to one or more gRNA recognition sequences within a target genomic locus in the CFD genomic nucleic acid molecule. The gRNA recognition sequence can include or be proximate to the start codon of a CFD genomic nucleic acid molecule or the stop codon of a CFD genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a CFD genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where Nis any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a CFD genomic nucleic acid molecule. An exemplary gRNA is a gRNA effective to direct a Cas enzyme to bind to or cleave a CFD genomic nucleic acid molecule, wherein the gRNA comprises a DNA-targeting segment that hybridizes to a gRNA recognition sequence within the CFD genomic nucleic acid molecule. Exemplary gRNAs comprise a DNA-targeting segment that hybridizes to a gRNA recognition sequence present within a CFD genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the CFD genomic nucleic acid molecule. The Cas protein can cleave the nucleic acid molecule at a site within or outside of the nucleic acid sequence present in the CFD genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the CFD genomic nucleic acid molecule to which a DNA-targeting segment of a gRNA will bind.

Such methods can result, for example, in a CFD genomic nucleic acid molecule in which a region of the CFD genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the CFD genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In any of the methods of treatment or prevention described herein, the subject being treated may comprise a CFD variant nucleic acid molecule. In some embodiments, the subject being treated is heterozygous for the CFD variant nucleic acid molecule. In some embodiments, the subject being treated is CFD reference. The CFD variant nucleic acid molecule can be any of the CFD variant nucleic acid molecules disclosed herein. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

In some embodiments, the methods of treatment or prevention further comprise detecting the presence or absence of a CFD variant nucleic acid molecule in a biological sample from the subject. In some embodiments, the CFD variant nucleic acid molecule can be any of the CFD variant nucleic acid molecules disclosed herein. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

The present disclosure also provides methods of treating a subject with a macular degeneration therapeutic agent or macular degeneration therapy that treats or inhibits macular degeneration, wherein the subject has macular degeneration or is at risk of developing macular degeneration. The methods comprise determining whether the subject has a CFD variant nucleic acid molecule by obtaining or having obtained a biological sample from the subject, and performing or having performed a sequence analysis on the biological sample to determine if the subject has a genotype comprising the CFD variant nucleic acid molecule. In embodiments where the subject is CFD reference, the methods further comprise administering or continuing to administer the macular degeneration therapeutic agent in an amount that is the same as or less than a standard dosage amount or macular degeneration therapy to the subject, and/or administering a CFD inhibitor to the subject. In embodiments where the subject is heterozygous for the CFD variant nucleic acid molecule, the methods further comprise administering or continuing to administer the macular degeneration therapeutic agent in an amount that is the same as or less than a standard dosage amount or macular degeneration therapy to the subject, and/or administering a CFD inhibitor to the subject. In embodiments where the subject is homozygous for the CFD variant nucleic acid molecule, the methods further comprise administering or continuing to administer the macular degeneration therapeutic agent in a standard dosage amount or macular degeneration therapy to the subject. The presence of a CFD variant nucleic acid molecule indicates the subject has a decreased risk of developing macular degeneration. In some embodiments, the subject is CFD reference. In some embodiments, the subject is heterozygous for a CFD variant nucleic acid molecule. In some embodiments, the subject is homozygous for a CFD variant nucleic acid molecule. In any of the embodiments described herein, the CFD inhibitor is an example of a macular degeneration therapeutic agent. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

For subjects that are genotyped or determined to be either CFD reference or heterozygous for a CFD variant nucleic acid molecule, such subjects can be administered a CFD inhibitor, as described herein.

Detecting the presence or absence of a CFD variant nucleic acid molecule in a biological sample from a subject and/or determining whether a subject has a CFD variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is CFD reference, the subject is administered a macular degeneration therapeutic agent in an amount that is the same as or less than a standard dosage amount or macular degeneration therapy, and/or a CFD inhibitor. In some embodiments, when the subject is heterozygous for a CFD variant nucleic acid molecule, the subject is administered a macular degeneration therapeutic agent in an amount that is the same as or less than a standard dosage amount or macular degeneration therapy, and/or a CFD inhibitor.

In some embodiments, the treatment or prevention methods comprise detecting the presence or absence of a decrease in the expression of a CFD variant mRNA or polypeptide in a biological sample from the subject. In some embodiments, when the subject does not have a decrease in the expression of a CFD variant mRNA or polypeptide, the subject is administered a macular degeneration therapeutic agent in an amount that is the same as or less than a standard dosage amount or macular degeneration therapy, and/or a CFD inhibitor. In some embodiments, when the subject has a decrease in the expression of a CFD variant mRNA or polypeptide, the subject is administered a macular degeneration therapeutic agent in a standard dosage amount or macular degeneration therapy.

The present disclosure also provides methods of treating a subject with a macular degeneration therapeutic agent or macular degeneration therapy that treats or inhibits macular degeneration, wherein the subject has macular degeneration or is at risk of developing macular degeneration. The methods comprise determining whether the subject has a decrease in the expression of a CFD variant mRNA or polypeptide by obtaining or having obtained a biological sample from the subject, and performing or having performed an assay on the biological sample to determine if the subject a decrease in the expression of a CFD variant mRNA or polypeptide. In embodiments where the subject does not have a decrease in the expression of a CFD variant mRNA or polypeptide, the methods further comprise administering or continuing to administer the macular degeneration therapeutic agent in an amount that is the same as or less than a standard dosage amount or macular degeneration therapy to the subject, and/or administering a CFD inhibitor to the subject. In embodiments where the subject has a decrease in the expression of a CFD variant mRNA or polypeptide, the methods further comprise administering or continuing to administer the macular degeneration therapeutic agent in a standard dosage amount or macular degeneration therapy to the subject. The presence of a decrease in the expression of a CFD variant mRNA or polypeptide indicates the subject has a decreased risk of developing macular degeneration. In some embodiments, the subject has a decrease in the expression of a CFD variant mRNA or polypeptide. In some embodiments, the subject does not have a decrease in the expression of a CFD variant mRNA or polypeptide. In any of the embodiments described herein, the CFD inhibitor is an example of a macular degeneration therapeutic agent. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

Detecting a decrease in the expression of a CFD variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In some embodiments, the treatment or prevention methods comprise detecting the presence or absence of a CFD variant polypeptide in a biological sample from the subject. In some embodiments, when the subject does not have a CFD variant polypeptide, the subject is administered a macular degeneration therapeutic agent in an amount that is the same as or less than a standard dosage amount or macular degeneration therapy, and/or a CFD inhibitor. In some embodiments, when the subject has a CFD variant polypeptide, the subject is administered a macular degeneration therapeutic agent in standard dosage amount or macular degeneration therapy.

The present disclosure also provides methods of treating a subject with a macular degeneration therapeutic agent or macular degeneration therapy that treats or inhibits macular degeneration, wherein the subject has macular degeneration or is at risk of developing macular degeneration. The methods comprise determining whether the subject has a CFD variant polypeptide by obtaining or having obtained a biological sample from the subject and performing or having performed an assay on the biological sample to determine if the subject has a CFD variant polypeptide. When the subject does not have a CFD variant polypeptide, the subject is administered the macular degeneration therapeutic agent in an amount that is the same as or less than a standard dosage amount or macular degeneration therapy, and/or a CFD inhibitor. When the subject has a CFD variant polypeptide, the subject is administered the macular degeneration therapeutic agent in a standard dosage amount or macular degeneration therapy. The presence of a CFD variant polypeptide indicates the subject has a decreased risk of developing macular degeneration. In some embodiments, the subject has a CFD variant polypeptide. In some embodiments, the subject does not have a CFD variant polypeptide.

The present disclosure also provides methods of preventing a subject from developing macular degeneration by administering a macular degeneration therapeutic agent or macular degeneration therapy that prevents macular degeneration. In some embodiments, the method comprises determining whether the subject has a CFD variant polypeptide by obtaining or having obtained a biological sample from the subject and performing or having performed an assay on the biological sample to determine if the subject has a CFD variant polypeptide. When the subject does not have a CFD variant polypeptide, the subject is administered the macular degeneration therapeutic agent in an amount that is the same as or less than a standard dosage amount or macular degeneration therapy, and/or a CFD inhibitor. When the subject has a CFD variant polypeptide, the subject is administered the macular degeneration therapeutic agent in a standard dosage amount or macular degeneration therapy. The presence of a CFD variant polypeptide indicates the subject has a decreased risk of developing macular degeneration. In some embodiments, the subject has a CFD variant polypeptide. In some embodiments, the subject does not have a CFD variant polypeptide.

Detecting the presence or absence of a CFD variant polypeptide in a biological sample from a subject and/or determining whether a subject has a CFD variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the CFD inhibitor is a small molecule. In some embodiments, the small molecule is low molecular weight (<900 daltons) organic compound. In some embodiments, the CFD inhibitor includes, but is not limited to danicopan (ACH-4471), danicopan analogs (ACH-5228 or ALXN-2050), BCX9930, BCX1470,nafamostat, and vemircopan. In some embodiments, the CFD inhibitor comprises danicopan (ACH-4471). In some embodiments, the CFD inhibitor comprises a danicopan analog (ACH-5228 or ALXN-2050). In some embodiments, the CFD inhibitor comprises BCX9930. In some embodiments, the CFD inhibitor comprises BCX1470. In some embodiments, the CFD inhibitor comprises nafamostat. In some embodiments, the CFD inhibitor comprises vemircopan. In some embodiments, the CFD inhibitor comprises a small molecule disclosed in PCT Publication WO2014/002051, U.S. Pat. No. 10,092,584, U.S. Patent Application Publication 2019/0345135, or PCT Publication WO2017/136395.

In some embodiments, the CFD inhibitor comprises an antibody, or antigen-binding fragment thereof. In some embodiments, the antibody, or antigen-binding fragment thereof, binds specifically to human CFD. In some embodiments, the antibody is a fully human monoclonal antibody (mAb), or antigen-binding fragment thereof, that specifically binds and neutralizes, inhibits, blocks, abrogates, reduces, or interferes with, at least one activity of CFD, in particular, human CFD. In some embodiments, an antibody or fragment thereof can neutralize, inhibit, block, abrogate, reduce, or interfere with, an activity of CFD by binding to an epitope of CFD that is directly involved in the targeted activity of CFD. In some embodiments, an antibody or fragment thereof can neutralize, inhibit, block, abrogate, reduce, or interfere with, an activity of CFD by binding to an epitope of CFD that is not directly involved in the targeted activity of CFD, but the antibody or fragment binding thereto sterically or conformationally inhibits, blocks, abrogates, reduces, or interferes with, the targeted activity of CFD. In some embodiments, an antibody or fragment thereof binds to an epitope of CFD that is not directly involved in the targeted activity of CFD (i.e., a non-blocking antibody), but the antibody or fragment binding thereto results in the enhancement of the clearance of CFD from the circulation, compared to the clearance of CFD in the absence of the antibody or fragment thereof, thereby indirectly inhibiting, blocking, abrogating, reducing, or interfering with, an activity of CFD. Clearance of CFD from the circulation can be particularly enhanced by combining two or more different non-blocking antibodies that do not compete with one another for specific binding to CFD. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933). In some embodiments, the antibody comprises lampalizumab.

In some embodiments, the antibody or antigen-binding fragment thereof specifically binds to CFD with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-CFD antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made in order to modify complement dependent cytotoxicity (CDC).

The present disclosure also provides compositions comprising a combination of an antibody or antigen-binding fragment thereof and a macular degeneration therapeutic agent.

In some embodiments, the macular degeneration therapeutic agents include, but are not limited to, aflibercept, ranibizumab, bevacizumab, pegaptanib, conbercept, brolucizumab, faricimab, nepafenac, verteporfin, ketorolac, lidocaine, pegcetacoplan, and avacincaptad pegol intravitreal solution. In some embodiments, the macular degeneration therapeutic agent comprises aflibercept. In some embodiments, the macular degeneration therapeutic agent comprises ranibizumab. In some embodiments, the macular degeneration therapeutic agent comprises bevacizumab. In some embodiments, the macular degeneration therapeutic agent comprises pegaptanib. In some embodiments, the macular degeneration therapeutic agent comprises conbercept. In some embodiments, the macular degeneration therapeutic agent comprises brolucizumab. In some embodiments, the macular degeneration therapeutic agent comprises faricimab. In some embodiments, the macular degeneration therapeutic agent comprises nepafenac. In some embodiments, the macular degeneration therapeutic agent comprises verteporfin. In some embodiments, the macular degeneration therapeutic agent comprises ketorolac. In some embodiments, the macular degeneration therapeutic agent comprises lidocaine. In some embodiments, the macular degeneration therapeutic agent comprises EMPAVELI® (pegcetacoplan). In some embodiments, the macular degeneration therapeutic agent comprises IZERVAY™ (avacincaptad pegol intravitreal solution). Additional macular degeneration therapies include any therapy used to reduce or manage macular degeneration risk factors. In some embodiments, the macular degeneration therapeutic agent or macular degeneration therapy can be combined with a CFD inhibitor. In some embodiments, the treatment therapy for macular degeneration is laser photocoagulation/photodynamic therapy (PDT), optionally in combination with verteporfin. These treatment therapies may be delayed or avoided altogether by treatment with a CFD inhibitor as described herein.

In some embodiments, the dose of the macular degeneration therapeutic agents that treat, prevent, or inhibit macular degeneration can be decreased by about 10%, by about 20%, by about 30%, by about 40%, by about 50%, by about 60%, by about 70%, by about 80%, or by about 90% for subjects that are heterozygous for a CFD variant nucleic acid molecule or CFD reference (i.e., a less than the standard dosage amount) compared to subjects that are homozygous for a CFD variant nucleic acid molecule (who may receive a standard dosage amount). In some embodiments, the dose of the macular degeneration therapeutic agents that treat, prevent, or inhibit macular degeneration can be decreased by about 10%, by about 20%, by about 30%, by about 40%, or by about 50%. In some embodiments, the dose of the macular degeneration therapeutic agents that treat, prevent, or inhibit macular degeneration can be decreased by about 10%, by about 20%, by about 30%, by about 40%, by about 50%, by about 60%, by about 70%, by about 80%, or by about 90% for subjects that are heterozygous for a CFD variant nucleic acid molecule or CFD reference compared to subjects that are CFD reference. In addition, subjects that are heterozygous for a CFD variant nucleic acid molecule or CFD reference can be administered the macular degeneration therapeutic agents less frequently compared to subjects that are heterozygous for the CFD variant nucleic acid molecule.

Administration of the macular degeneration therapeutic agents that treat, prevent, or inhibit macular degeneration and/or CFD inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the macular degeneration therapeutic agents and/or CFD inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in macular degeneration, a decrease/reduction in the severity of macular degeneration (such as, for example, a reduction or inhibition of development of macular degeneration), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to macular degeneration, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of macular degeneration development/progression (such as, for example, a complete or partial avoidance/inhibition or a delay), and an increased survival time of the affected host animal, following administration of a therapeutic protocol. Treatment of macular degeneration encompasses the treatment of a subject already diagnosed as having any form of macular degeneration at any clinical stage or manifestation, the delay of the onset or evolution or aggravation or deterioration of the symptoms or signs of macular degeneration, and/or preventing and/or reducing the severity of macular degeneration.

In some embodiments, the CFD inhibitor and the macular degeneration therapeutic agent are disposed within a pharmaceutical composition. In some embodiments, the CFD inhibitor is disposed within a first pharmaceutical composition and the macular degeneration therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

The present disclosure also provides methods of identifying a subject having an increased risk of developing macular degeneration. In some embodiments, the method comprises determining or having determined in a biological sample obtained from the subject the presence or absence of a CFD variant nucleic acid molecule (such as a genomic nucleic acid molecule, mRNA molecule, and/or cDNA molecule). When the subject lacks a CFD variant nucleic acid molecule (i.e., the subject is genotypically categorized as CFD reference), then the subject has an increased risk of developing macular degeneration. When the subject has a CFD variant nucleic acid molecule (i.e., the subject is heterozygous or homozygous for a CFD variant nucleic acid molecule), then the subject has a decreased risk of developing macular degeneration. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

Having a single copy of a CFD variant nucleic acid molecule is more protective of a subject from developing macular degeneration than having no copies of a CFD variant nucleic acid molecule. Without intending to be limited to any particular theory or mechanism of action, it is believed that a single copy of a CFD variant nucleic acid molecule (i.e., heterozygous for a CFD variant nucleic acid molecule) is protective of a subject from developing macular degeneration and it is also believed that having two copies of a CFD variant nucleic acid molecule (i.e., homozygous for a CFD variant nucleic acid molecule) may be more protective of a subject from developing macular degeneration, relative to a subject with a single copy. Thus, in some embodiments, a single copy of a CFD variant nucleic acid molecule may not be completely protective, but instead, may be partially or incompletely protective of a subject from developing macular degeneration. While not desiring to be bound by any particular theory, there may be additional factors or molecules involved in the development of macular degeneration that are still present in a subject having a single copy of a CFD variant nucleic acid molecule, thus resulting in less than complete protection from the development of macular degeneration.

Determining whether a subject has a CFD variant nucleic acid molecule in a biological sample from a subject and/or determining whether a subject has a CFD variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing macular degeneration, the subject is administered a macular degeneration therapeutic agent or macular degeneration therapy, and/or a CFD inhibitor, as described herein. For example, when the subject is CFD reference, and therefore has an increased risk of developing macular degeneration, the subject is administered a macular degeneration therapeutic agent in an amount that is the same as or less than a standard dosage amount or macular degeneration therapy, and/or is administered a CFD inhibitor. In some embodiments, when the subject is heterozygous for a CFD variant nucleic acid molecule, the subject is administered the macular degeneration therapeutic agent in an amount that is the same as or less than a standard dosage amount or macular degeneration therapy, and/or is administered a CFD inhibitor. In some embodiments, when the subject is homozygous for a CFD variant nucleic acid molecule, the subject is administered a macular degeneration therapeutic agent in a standard dosage amount or macular degeneration therapy. In some embodiments, the subject is CFD reference. In some embodiments, the subject is heterozygous for a CFD variant nucleic acid molecule. In some embodiments, the subject is homozygous for a CFD variant nucleic acid molecule.

The present disclosure also provides methods of determining a subject's aggregate burden, or risk score, of having two or more CFD variant nucleic acid molecules, and/or two or more CFD variant polypeptides associated with a decreased risk of developing macular degeneration. The aggregate burden is the sum of two or more genetic variants that can be carried out in an association analysis with macular degeneration. In some embodiments, the subject is homozygous for one or more CFD variant nucleic acid molecules associated with a decreased risk of developing macular degeneration. In some embodiments, the subject is heterozygous for one or more CFD variant nucleic acid molecules associated with a decreased risk of developing macular degeneration. When the subject has a lower aggregate burden, the subject has an increased risk of developing macular degeneration, and the subject is administered or continued to be administered the macular degeneration therapeutic agent in an amount that is the same as or less than the standard dosage amount or macular degeneration therapy, and/or a CFD inhibitor. When the subject has a higher aggregate burden, the subject has a decreased risk of developing macular degeneration and the subject is administered or continued to be administered the macular degeneration therapeutic agent in a standard dosage amount or macular degeneration therapy. The higher the aggregate burden, the lower the risk of developing macular degeneration.

In some embodiments, a subject's aggregate burden of having any two or more CFD variant nucleic acid molecules represents a weighted sum of a plurality of any of the CFD variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the CFD gene, where the genetic burden is the number of alleles multiplied by the association estimate with macular degeneration or related outcome for each allele (e.g., a weighted polygenic burden score). In some embodiments, when the subject has an aggregate burden higher than a desired threshold score, the subject has a decreased risk of developing macular degeneration. In some embodiments, when the subject has an aggregate burden lower than a desired threshold score, the subject has an increased risk of developing macular degeneration.

In some embodiments, the aggregate burden may be divided into quintiles, e.g., top quintile, second quintile, intermediate quintile, fourth quintile, and bottom quintile, wherein the top quintile of aggregate burden corresponds to the lowest risk group and the bottom quintile of aggregate burden corresponds to the highest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with macular degeneration in the top 10%, top 20%, top 30%, top 40%, or top 50% of p-value range for the association. In some embodiments, each of the identified genetic variants comprise the genetic variants having association with macular degeneration with p-value of no more than about 10-2, about 10-3, about 10-4, about 10-5, about 10-6, about 10-7, about 10-8, about 10-9, about 10-10, about 10-11, about 10-12, about 10-13, about 10-14, about or 10-15. In some embodiments, the identified genetic variants comprise the genetic variants having association with macular degeneration with p-value of less than 5×10⁻⁸. In some embodiments, the identified genetic variants comprise genetic variants having association with macular degeneration in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the bottom decile, quintile, or tertile in a reference population. The threshold of the aggregate burden can be determined on the basis of the nature of the intended practical application and the risk difference that would be considered meaningful for that practical application.

In embodiments where the aggregate burden is determined for CFD genetic variants associated with macular degeneration, then the aggregate burden represents a subject's risk score for developing macular degeneration. In some embodiments, the aggregate burden or risk score includes the CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule. In some embodiments, a subject's aggregate burden can be determined for CFD genetic variants associated with macular degeneration in combination with additional genetic variants for other genes also associated with macular degeneration to produce a polygenic risk score (PRS) for developing macular degeneration. In some embodiments, the PRS includes the CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule. In some embodiments, a subject's aggregate burden can be determined for CFD genetic variants associated with macular degeneration in combination with, for example, complement factor H (CFH) genetic variants associated with macular degeneration, such as for example, Y402H, to produce a PRS for developing macular degeneration.

The present disclosure also provides methods of detecting the presence or absence of a CFD variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival cervicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any CFD variant nucleic acid molecule, preliminary processing designed to isolate or enrich the biological sample for the genomic DNA can be employed. A variety of techniques may be used for this purpose. When detecting the level of any CFD variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting a CFD variant nucleic acid molecule in a subject comprises performing a sequence analysis on a biological sample obtained from the subject to determine whether a CFD genomic nucleic acid molecule in the biological sample, and/or a CFD mRNA molecule in the biological sample, and/or a CFD cDNA molecule produced from an mRNA molecule in the biological sample, is present in the sample. In some embodiments, the methods detect the CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or an mRNA molecule produced therefrom, or a cDNA molecule produced from the mRNA molecule.

In some embodiments, the methods of detecting the presence or absence of a CFD variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a CFD genomic nucleic acid molecule or mRNA molecule, and if mRNA, optionally reverse transcribing the mRNA into cDNA. Such assays can comprise, for example determining the identity of these positions of the particular CFD nucleic acid molecule. In some embodiments, the method is an in vitro method.

In some embodiments, the determining step, detecting step, or sequence analysis comprises sequencing at least a portion of the nucleotide sequence of the CFD genomic nucleic acid molecule, the CFD mRNA molecule, or the CFD cDNA molecule in the biological sample that comprises a genetic variation compared to the corresponding CFD reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only a CFD genomic nucleic acid molecule is analyzed. In some embodiments, only a CFD mRNA is analyzed. In some embodiments, only a CFD cDNA obtained from the CFD mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a CFD variant genomic sequence, variant mRNA sequence, or variant cDNA sequence and not the corresponding CFD reference sequence under stringent conditions and determining whether hybridization has occurred.

In some embodiments, the determining step, detecting step, or sequence analysis comprises: a) amplifying at least a portion of the CFD nucleic acid molecule that encodes the CFD polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a CFD variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2× SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to CFD variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to CFD variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the CFD variant nucleic acid molecules disclosed herein. The primers described herein can be used to amplify any CFD variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding a CFD reference genomic nucleic acid molecule, a CFD reference mRNA molecule, and/or a CFD reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides macular degeneration therapeutic agents that treat, prevent, or inhibit macular degeneration for use in the treatment or prevention of macular degeneration in a subject having a CFD variant nucleic acid molecule. Any of the macular degeneration therapeutic agents that treat, prevent, or inhibit macular degeneration described herein can be used herein. Any of the CFD variant nucleic acid molecules disclosed herein can be used herein. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

The present disclosure also provides use of macular degeneration therapeutic agents that treat, prevent, or inhibit macular degeneration in the preparation of a medicament for treating or preventing macular degeneration in a subject having a CFD variant nucleic acid molecule. Any of the macular degeneration therapeutic agents that treat, prevent, or inhibit macular degeneration described herein can be used herein. Any of the CFD variant nucleic acid molecules disclosed herein can be used herein. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:860714:C:A (Cys51Ter), 19:863142:CACCTCGGGCTCGCGCGT:C (Thr223LeufsTer115), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

The present disclosure also provides CFD inhibitors for use in the treatment or prevention of macular degeneration in a subject that is CFD reference or is heterozygous for a CFD variant nucleic acid molecule. Any of the CFD inhibitors described herein can be used herein. Any of the CFD variant nucleic acid molecules disclosed herein can be used herein. In some embodiments, the CFD variant nucleic acid molecule is a CFD variant genomic nucleic acid molecule that comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:860714:C:A (Cys51Ter), 19:863142:CACCTCGGGCTCGCGCGT:C (Thr223LeufsTer115), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

The present disclosure also provides use of CFD inhibitors in the preparation of a medicament for treating or preventing macular degeneration in a subject that is CFD reference or is heterozygous for a CFD variant nucleic acid molecule. Any of the CFD inhibitors described herein can be used herein. Any of the CFD variant nucleic acid molecules disclosed herein can be used herein. In some embodiments, the CFD variant nucleic acid molecule is a CFD variant genomic nucleic acid molecule that comprises the genetic variation rs35186399 (19:860766:G:A; an Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter), or is an mRNA molecule produced therefrom, or is a cDNA molecule produced from the mRNA molecule.

In some embodiments, the CFD inhibitor and the macular degeneration therapeutic agent are disposed within a pharmaceutical composition. In some embodiments, the CFD inhibitor is disposed within a first pharmaceutical composition and the macular degeneration therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methods

An exome wide association analysis was performed to discover genetic variants affecting risk for age-related macular degeneration. DNA was extracted from whole blood and/or saliva samples from participants from biobanks and electronic health record cohorts recruited by collaborators of the Regeneron Genetics Center. These participating cohorts include the United Kingdom Biobank (UKB) cohort, the DiscoverEHR cohort from the Geisinger Health System (GHS) MyCode Community Health Initiative, Mount Sinai BioMe Biobank cohort (SINAI), The University of Pennsylvania Penn Medicine BioBank (UPENN-PMBB), Malmo Diet and Cancer Study (MDCS), and Biobanks at the University of Colorado, the University of California at Los Angeles, and Indiana University. DNA was sheared and hybridized to an exome capture kit, which removes most DNA that is not protein coding. DNA libraries were prepared for sequencing on a standard Illumina DNA sequencer. Sequence data was mapped to the reference genome and variant sequences were identified for all participants. These variant sequences were utilized in standard genome-wide and exome-wide statistical analyses to identify associations with phenotypes using the software Regenie.

Phenotypes were defined by international classification of diseases (ICD) 10 codes related to clinician diagnoses of age-related macular degeneration. Table 1 shows that the association between an aggregate of rare variants (<5%) predicted to cause loss-of-function (as described herein) in the CFD transcripts or proteins. Table 2 shows that these associations split into a top variant and the signal remain from other variants. Table 3 show that the signal across the top missense variant and all predicted loss-of-function (pLoF) variants.

### Example 2: Gene Level Association of CFD Protection From Macular Degeneration

Table 1 shows CFD variants grouped by function having a protective effect for macular degeneration. GENE P combines evidence across multiple frequency threshold and burden/gene tests using the aggregated Cauchy combination test (ACAT). pLoF=probable loss-of-function variants are classified by grouping predicted stop_gained, frameshift, splice acceptor, and splice donor variants on transcript ENST00000327726.11.

TABLE 1
CFD Burden test (Gene P = 9.4E−8)

| | Allele | Minor | | |
|---|---|---|---|---|
| Best | Frequesncy | Allele | | |
| Burden Tests | Threshold | Count | Effect | P value |
| pLoF | 0.001 | 485 | 0.82 | 0.49 |
| any missense | 0.05 | 22945 | 0.78 | 1.78E−09 |
| pLoF + any | 0.05 | 23428 | 0.78 | 1.35E−09 |
| missense | | | | |

| | Case Info | Control Info |
|---|---|---|
| Best Burden Tests | (Ref\|Alt\|Hom) | (Ref\|Alt\|Hom) |
| pLoF | 25708\|10\|0 | 784554\|475\|0 |
| any missense | 25650\|589\|2 | 777124\|22172\|90 |
| pLoF + any | 25639\|600\|2 | 776652\|22644\|90 |
| missense | | |

Table 2 shows the residual protective signal after removing the top missense variant.

TABLE 2

| | Selected | Frequency | | | |
|---|---|---|---|---|---|
| | Burden Test | Cutoff | Frequency | Effect | P |
| CFD | pLoF + any | 0.05 | 0.0142 | 0.78 | 1.35E−09 |
| | missense | | | | |
| | Any Missense | 0.05 | 0.0139 | 0.78* | 1.78E−09 |
| | pLoF only | 0.001 | 0.0003 | 0.82 | 0.49 |
| 19:860766:G:A | — | — | 0.011 | 0.79 | 7.78E−07 |
| (top variant | | | | (homozygous | |
| protecting | | | | OR = 0.67 | |
| against AMD) | | | | | |
| CFD with top | pLoF + any | 0.01 | 0.003 | 0.75 | 5.30E−4 |
| variant | missense | | | | |
| removed | without top | | | | |
| | variant | | | | |

| | Selected Burden | Cases | |
|---|---|---|---|
| | Test | (ref\|het\|hom) | Controls (ref\|het\|hom) |
| CFD | pLoF + any | 25639\|600\|2 | 776652\|22644\|90 |
| | missense | | |
| | Any Missense | 25650\|589\|2 | 777124\|22172\|90 |
| | pLoF only | 25708\|10\|0 | 784554\|475\|0 |
| 19:860766:G:A | — | 25768\|471\|2 | 781991\|17308\|86 |
| (top variant | | | |
| protecting against | | | |
| AMD) | | | |
| CFD with top | pLoF + any | 26112\|129\|0 | 794025\|5357\|4 |
| variant removed | missense without | | |
| | top variant | | |

Table 3 shows evidence across the top missense and all supporting probable loss-of-function (pLoF) variants.

TABLE 3

| Name | Effect | Pval | AAF |
|---|---|---|---|
| 19:860766:G:A | 7.91 × 10−1 | 7.78 × 10−7 | 1.09 × 10−2 |
| 19:860741:G:A | 2.52 × 101 | 1.18 × 10−1 | 1.86 × 10−5 |
| 19:860775:T:G | 4.88 × 10−1 | 2.46 × 10−1 | 7.81 × 10−5 |
| 19:863105:GC:G | 3.15 × 10−1 | 4.22 × 10−1 | 2.76 × 10−5 |
| 19:860933:C:A | 5.74 × 10−1 | 4.57 × 10−1 | 5.95 × 10−5 |
| 19:861912:G:T | 3.54 × 10−1 | 4.80 × 10−1 | 2.90 × 10−5 |
| 19:863142:CACCTCGGGCTCGCGCGT:C | 3.57 × 10−1 | 5.50 × 10−1 | 3.13 × 10−5 |
| 19:860714:C:A | 2.06 | 5.86 × 10−1 | 3.60 × 10−5 |
| 19:861785:C:A | 3.38 × 10−1 | 6.20 × 10−1 | 6.96 × 10−6 |
| 19:861926:C:A | 3.59 × 10−1 | 6.33 × 10−1 | 1.97 × 10−5 |
| 19:860692:AG:A | 3.55 × 10−1 | 6.61 × 10−1 | 1.28 × 10−5 |
| 19:861826:GCCCGGACAGC:G | 3.54 × 10−1 | 6.77 × 10−1 | 9.28 × 10−6 |
| 19:860752:CG:C | 3.47 × 10−1 | 6.85 × 10−1 | 2.35 × 10−5 |
| 19:859745:G:T | 3.61 × 10−1 | 7.50 × 10−1 | 1.04 × 10−5 |
| 19:860686:C:A | 8.52 × 10−1 | 8.72 × 10−1 | 3.89 × 10−5 |
| Name | Case_Info | Control_Info | varianteffect |
| 19:860766:G:A | 25,768 \| 471 \| 2 | 781,991 \| 17,308 \| | missense |
| | | 86 | |
| 19:860741:G:A | 6,965 \| 1 \| 0 | 424,007 \| 15 \| 0 | stop_gained |
| 19:860775:T:G | 21,855 \| 1 \| 0 | 694,701 \| 111 \| 0 | splice_donor |
| 19:863105:GC:G | 8,140 \| 0 \| 0 | 100,551 \| 6 \| 0 | frameshift |
| 19:860933:C:A | 13,143 \| 1 \| 0 | 566,474 \| 68 \| 0 | stop_gained |
| 19:861912:G:T | 6,966 \| 0 \| 0 | 423,982 \| 25 \| 0 | stop_gained |
| 19:863142:CACCTCGGGCTCGCGCGT:C | 6,964 \| 0 \| 0 | 423,930 \| 27 \| 0 | frameshift |
| 19:860714:C:A | 6,965 \| 1 \| 0 | 423,990 \| 30 \| 0 | stop_gained |
| 19:861785:C:A | 6,966 \| 0 \| 0 | 424,016 \| 6 \| 0 | stop_gained |
| 19:861926:C:A | 6,966 \| 0 \| 0 | 424,005 \| 17 \| 0 | stop_gained |
| 19:860692:AG:A | 6,966 \| 0 \| 0 | 424,004 \| 11 \| 0 | frameshift |
| 19:861826:GCCCGGACAGC:G | 6,966 \| 0 \| 0 | 423,982 \| 8 \| 0 | frameshift |
| 19:860752:CG:C | 6,176 \| 0 \| 0 | 142,493 \| 7 \| 0 | frameshift |
| 19:859745:G:T | 6,966 \| 0 \| 0 | 424,013 \| 9 \| 0 | splice donor |
| 19:860686:C:A | 14,317 \| 1 \| 0 | 243,053 \| 19 \| 0 | stop_gained |

| Name | hgvsc | Hgvsp |
|---|---|---|
| 19:860766:G:A | ENST00000327726.11:c.205G>A | ENSP00000332139.4:p.Glu69Lys |
| 19:860741:G:A | ENST00000327726.11:c.180G>A | ENSP00000332139.4:p.Trp60Ter |
| 19:860775:T:G | ENST00000327726.11:c.212+2T>G | NA |
| 19:863105:GC:G | ENST00000327726.11:c.632del | ENSP00000332139.4:p.Pro211Arg |
| | | fsTer101 |
| 19:860933:C:A | ENST00000327726.11:c.285C>A | ENSP00000332139.4:p.Tyr95Ter |
| 19:861912:G:T | ENST00000327726.11:c.571G>T | ENSP00000332139.4:p.Glu191Ter |
| 19:863142:CACCTCG | ENST00000327726.11:c.667_683del | ENSP00000332139.4:p.Thr223Leu |
| GGCTCGCGCGT:C | | fsTer115 |
| 19:860714:C:A | ENST00000327726.11:c.153C>A | ENSP00000332139.4:p.Cys51Ter |
| 19:861785:C:A | ENST00000327726.11:c.444C>A | ENSP00000332139.4:p.Cys148Ter |
| 19:861926:C:A | ENST00000327726.11:c.585C>A | ENSP00000332139.4:p.Cys195Ter |
| 19:860692:AG:A | ENST00000327726.11:c.132del | ENSP00000332139.4:p.GIn44Hisfs |
| | | Ter2 |
| 19:861826:GCCCGGA | ENST00000327726.11:c.487_496del | ENSP00000332139.4:p.Pro163Cys |
| CAGC:G | | fsTer28 |
| 19:860752:CG:C | ENST00000327726.11:c.193del | ENSP00000332139.4:p.Ala65Argf |
| | | sTer91 |
| 19:859745:G:T | ENST00000327726.11:c.55+1G>T | NA |
| 19:860686:C:A | ENST00000327726.11:c.125C>A | ENSP00000332139.4:p.Ser42Ter |

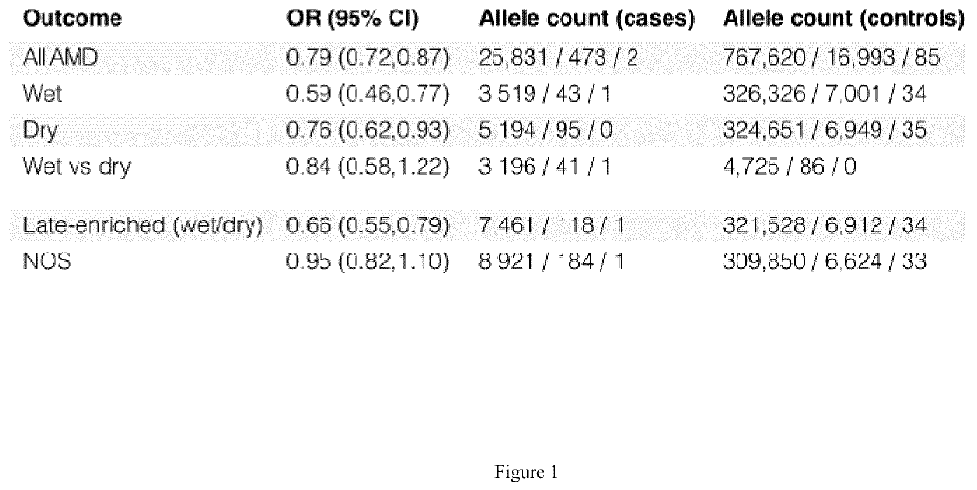

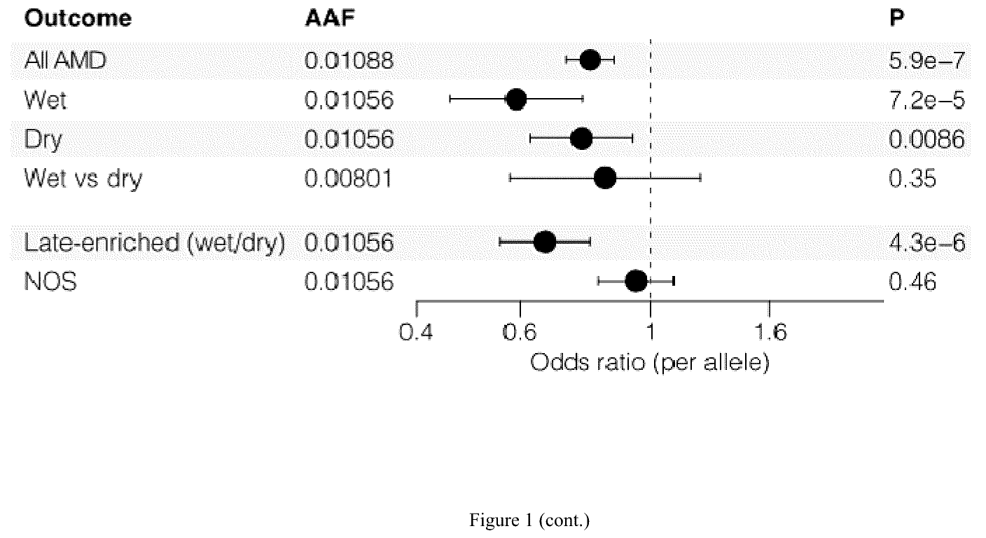

FIG. 1 shows analysis results for wet and dry AMD.

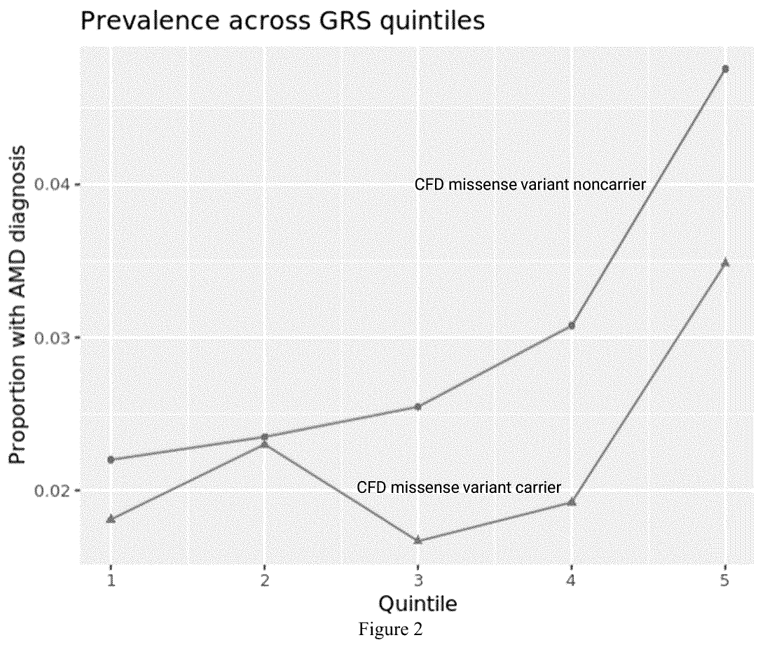

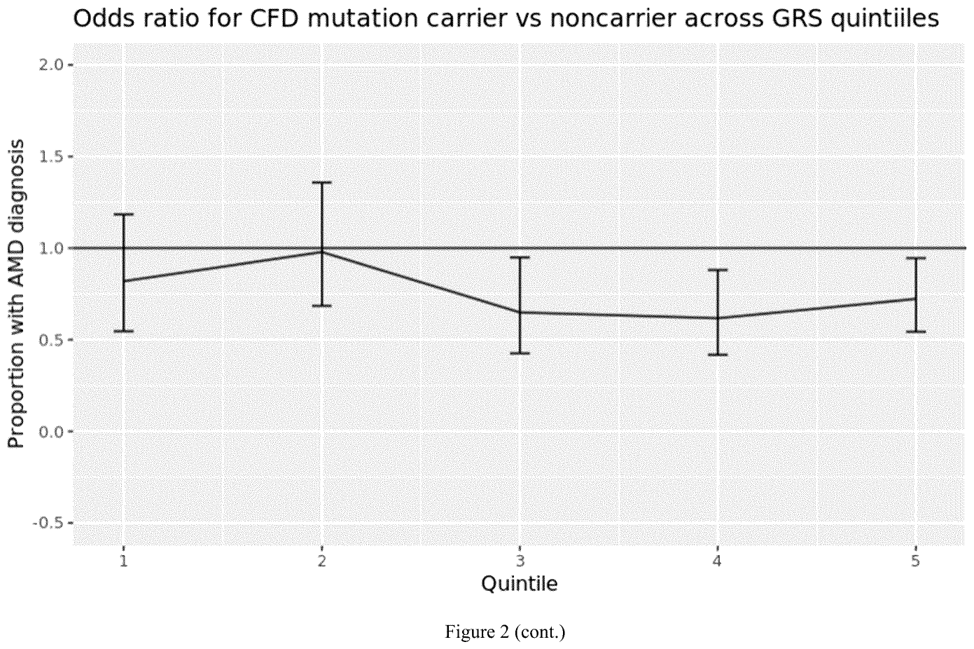

FIG. 2 shows CFD missense variant modifies risk in individuals with high genetic risk for AMD based on genetic risk score stratification. Referring to FIG. 2, Quintile=CFD missense variant carrier (cases/controls) (1=29/1573; 2=37/1571; 3=27/1592; 4=31/1582; and 5=56/1551); and CFD missense variant noncarrier (cases/controls) (1=3653/162376; 2=3902/162135; 3=4231/161892; 4=5114/161101; and 5=7896/158095). Comparing prevalence stratified by GRS from all variants in Gorman et al., medRxiv 2022.08.16.22278855 (world wide web at “doi.org/10.1101/2022.08.16.22278855”) after separating out CFD protective missense variant carriers from noncarriers shows that the CFD protective effect is strongest for the top 60% with overall genetic risk.

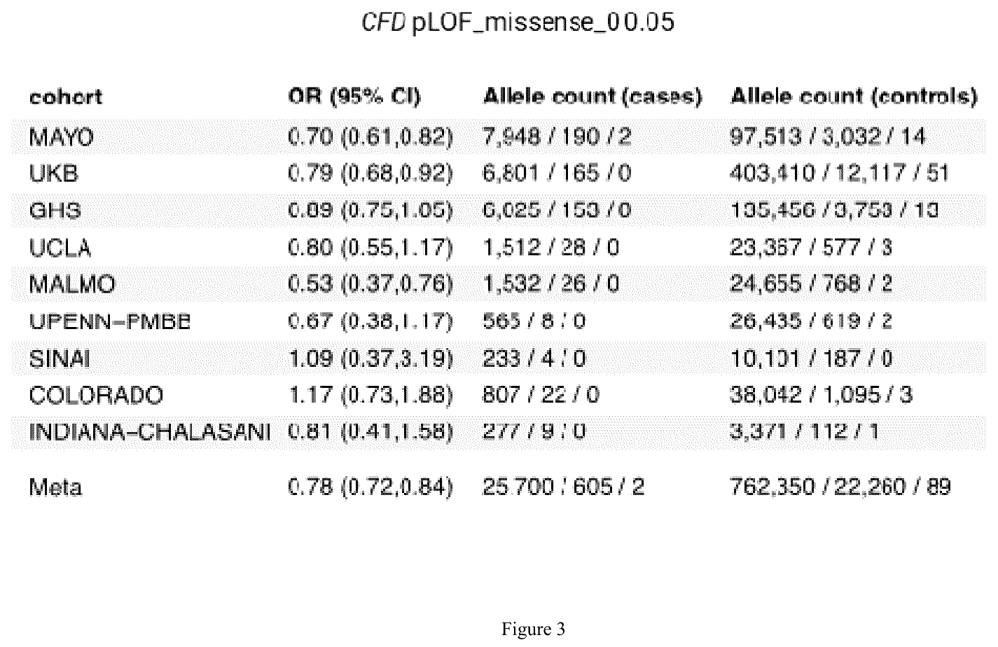

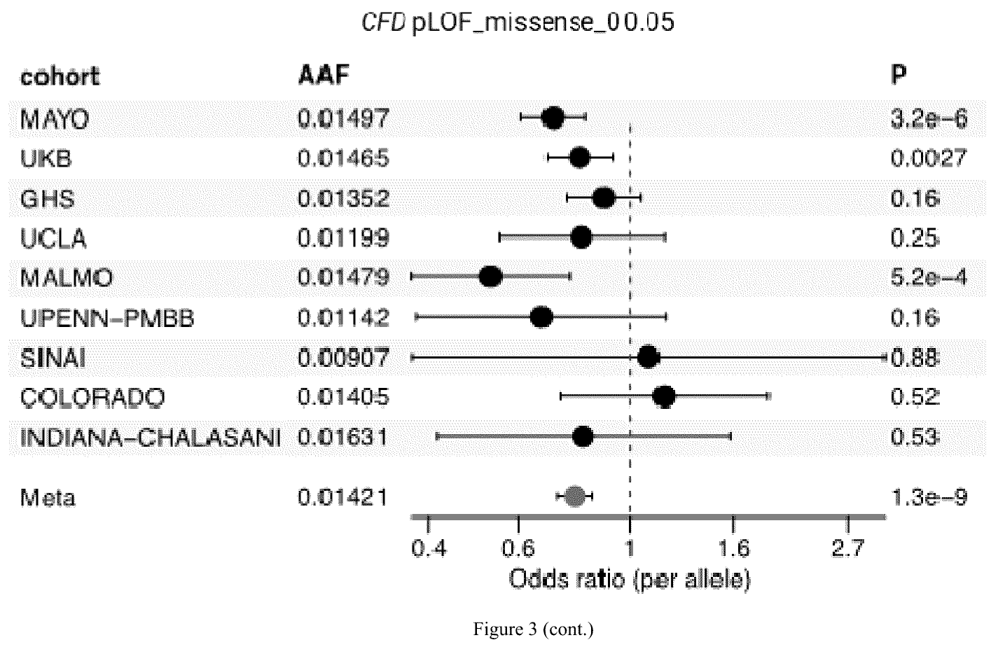

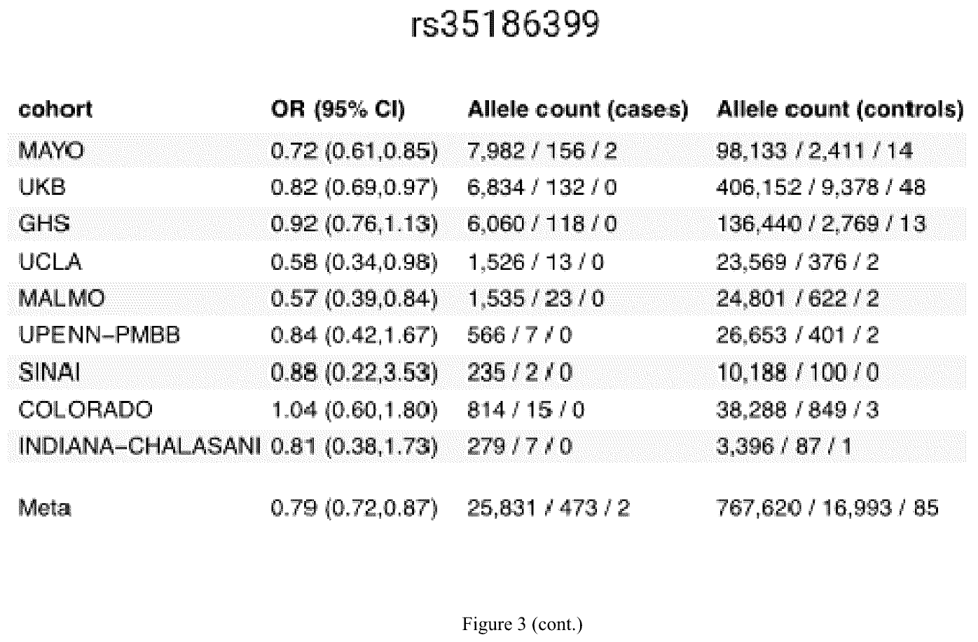

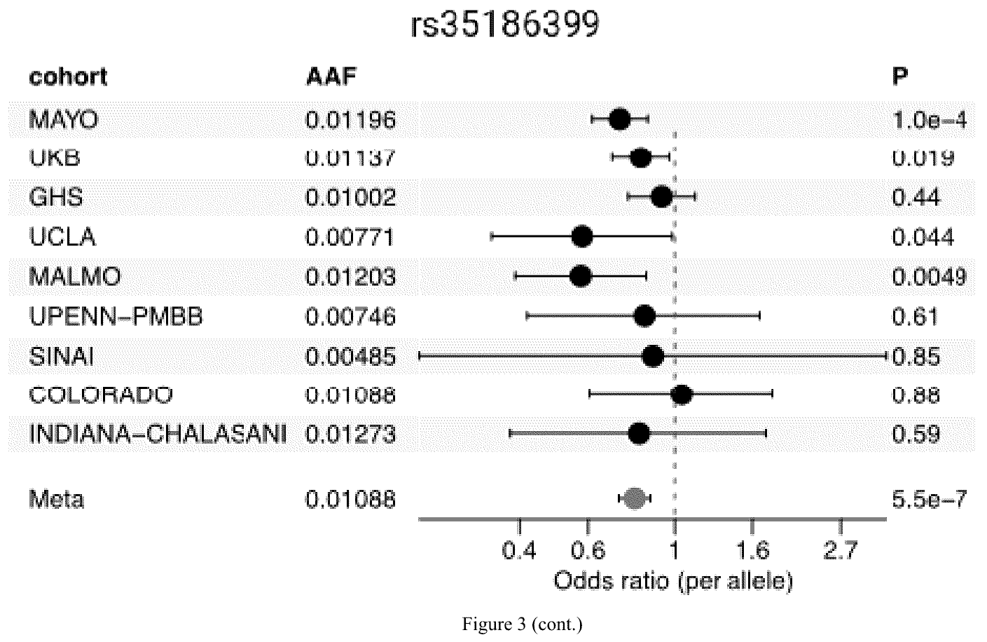

FIG. 3 shows CFD protective signal consistent in largest cohorts for burden mask and top variant. There is a protective effect for both wet and dry AMD.

### Example 3: Humans Carrying the CFD E69K Mutation Have Lower AMD Risk

Genome-wide association analysis of common and rare exonic variants in individuals who have diagnoses of AMD and controls was performed using nine cohorts. Four cohorts had diagnoses of late AMD available for evaluation. Association analysis was performed by using Regenie, which accounts for population structure and enables aggregate testing of mutations in genes (burden testing). A meta-analysis was performed across cohorts. Results are shown in Table 4.

TABLE 4

| | | Minor Allele | | |
|---|---|---|---|---|
| All AMD | Burden Mask | Frequency | Odds-ratio | P |
| CFD | pLoF* + Any Missense | 0.05 | 0.78 | 1.3E−9 |
| | Any Missense | 0.05 | 0.78* | 1.8E−9 |
| | pLoF | 0.001 | 0.82 | 0.47 |
| | — | 0.011 | 0.79 | 5.9E−7 |

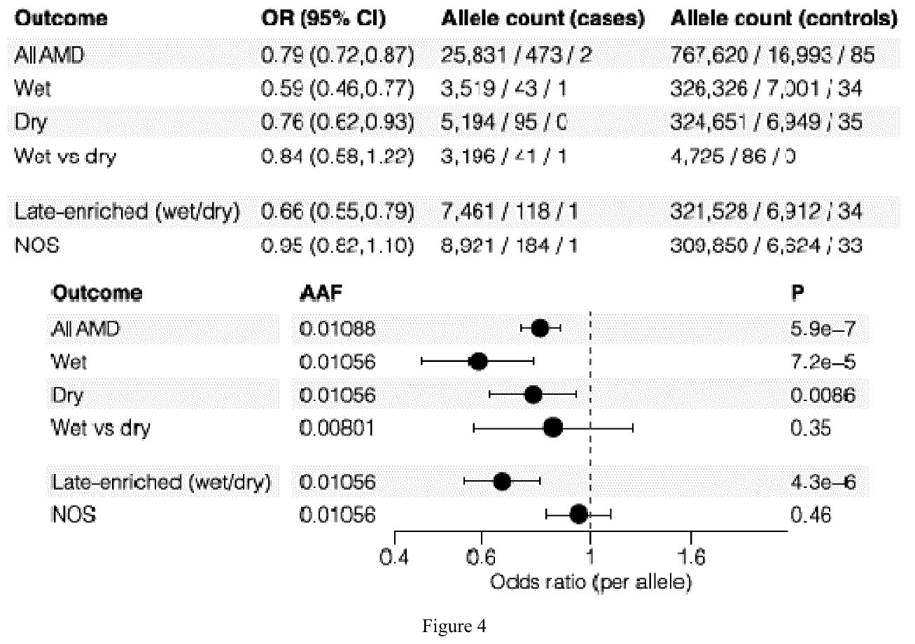

*pLoF=probable loss-of-function mutations, including stop gain, frameshift, splice donor, splice acceptor.
The top variant, 19:860766:G:A (E69K) showed consistent protection for AMD and AMD subtypes (see, FIG. 4).

### Example 4: E69K Mutation can Reduce Risk From the Most Prevalent Cause of AMD

CFH is an inhibitor of the alternative pathway of complement signaling. A common missense variant results in the CFH Y402H mutation, which is associated with reduced CFH activity, elevated alternative pathway activity, and elevated AMD risk. The GWAS signal near CFH accounts for about 10% of all AMD risk.

No protection from CFD wild type (GG) showed increasing risk from additional copies of CFH Y402H: OR changes from 1→1.4→1.9. One protective variant from CFD (E69K, GA) reduced risk from CFH Y402H: OR changes from 0.95→1.1→1.1. The “flattening” of risk suggested that this mutation is sufficient to block AP signaling. Two protective variants suggested even larger effect.

P values were computed by stratifying data across the Y402H mutations and computing the protective effect of CFD E69K within strat to test the “gene by gene” interaction: 1) No Y402H: OR=0.97; P-value=0.52; 2) One Y402H: OR=0.76; P-value=0.0013; and 3: Two Y402H: OR=0.58; P-value=7.8e−7.

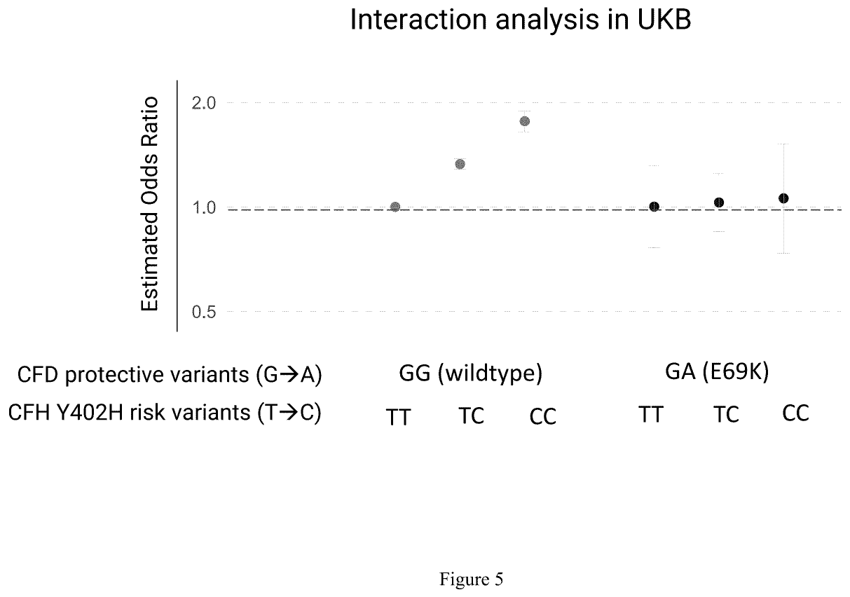

Results are shown in FIG. 5.

### Example 5: CFD mRNA and Protein Expression in Human Eye

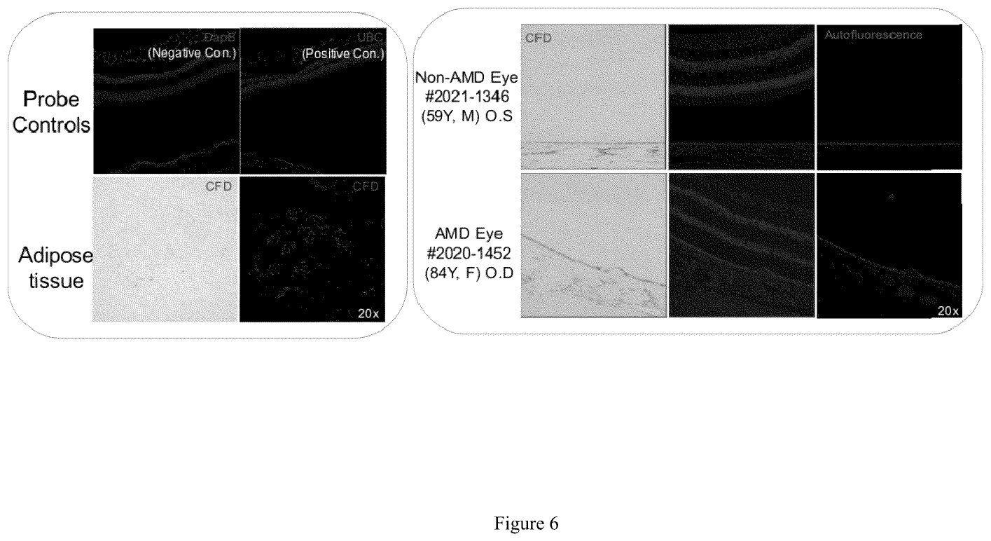

Human eye single-cell RNA-sequencing showed choroidal cell expression. Cfd in situ hybridization confirmed the expression in neural retina (in microglia) and choroid (Schwann cells and fibroblasts) (see, FIG. 6). CFD mRNA was significantly upregulated in the choroid relative to that of the retina and RPE. Additionally, in situ hybridization showed similar expression patterns in across normal and AMD-affected eyes, with expression in the neural retina and choroid cells and absent in the RPE. Adipose tissue, where CFD is known to be expressed, is provided as a positive control.

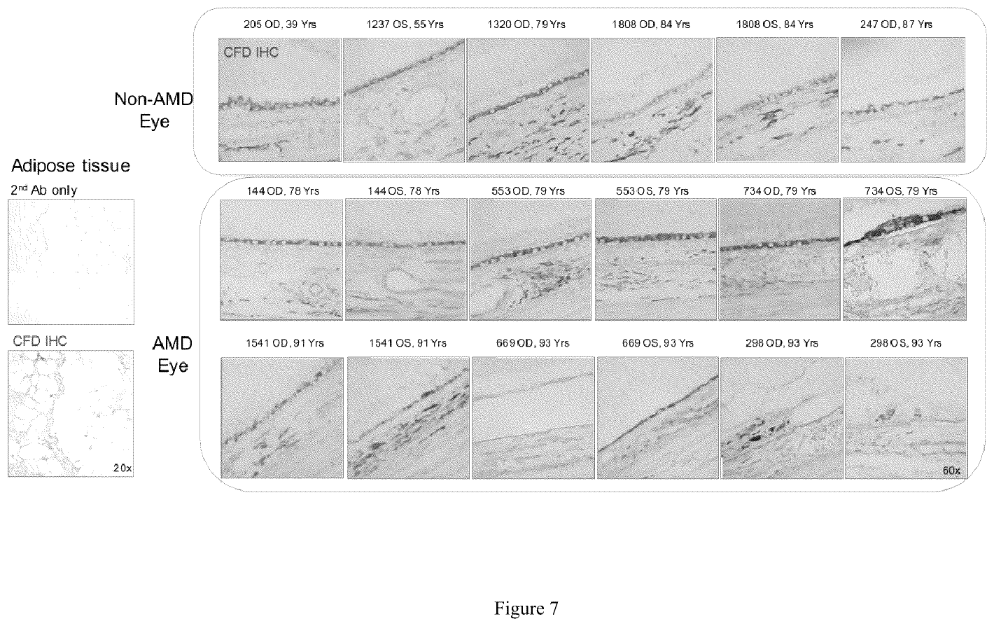

CFD protein was found in retinal pigment epithelium cells and choroidal tissues by immunohistochemistry (see, FIG. 7). Human eye sections showed CFD protein in RPE, choroidal, and sclera tissues. No differences were found in AMD and non-AMD eyes. Human adipose tissue was used as positive control. CFD protein was identified not only in the choroid, choriocapillaris, but also RPE cells. These data suggest that CFD in the eye may arise from both local and distal sites of expression however, in post-symptomatic AMD eyes, there was no obvious increase in CFD deposition.

### Example 6: Variant Characterization With In Vitro C3 Convertase Assay

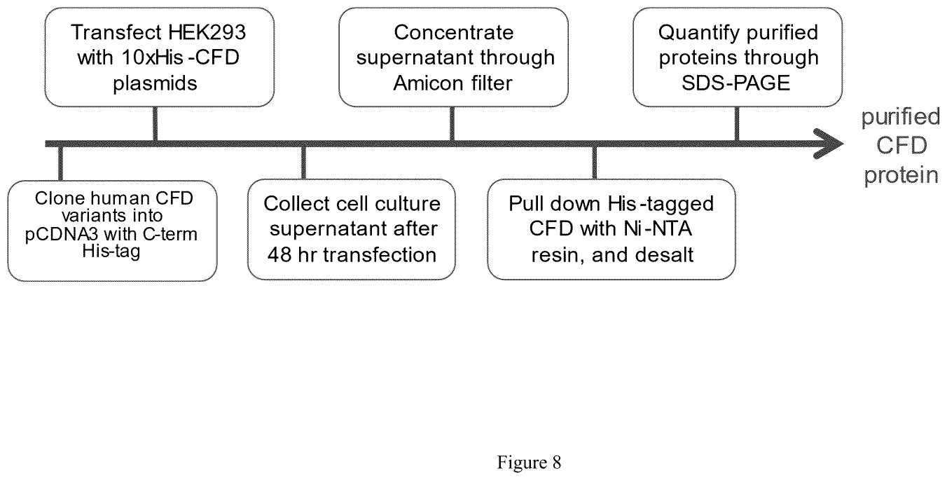

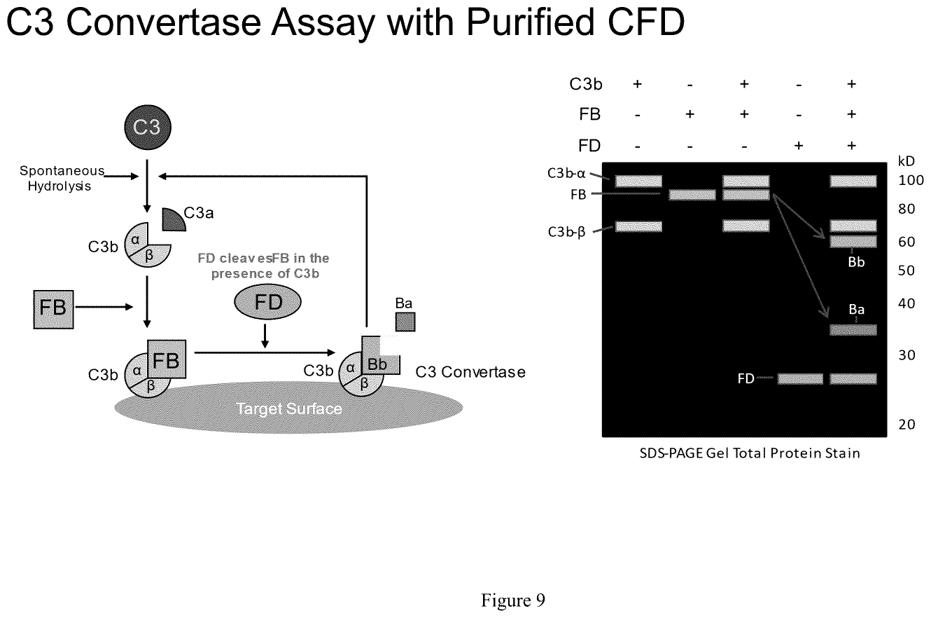

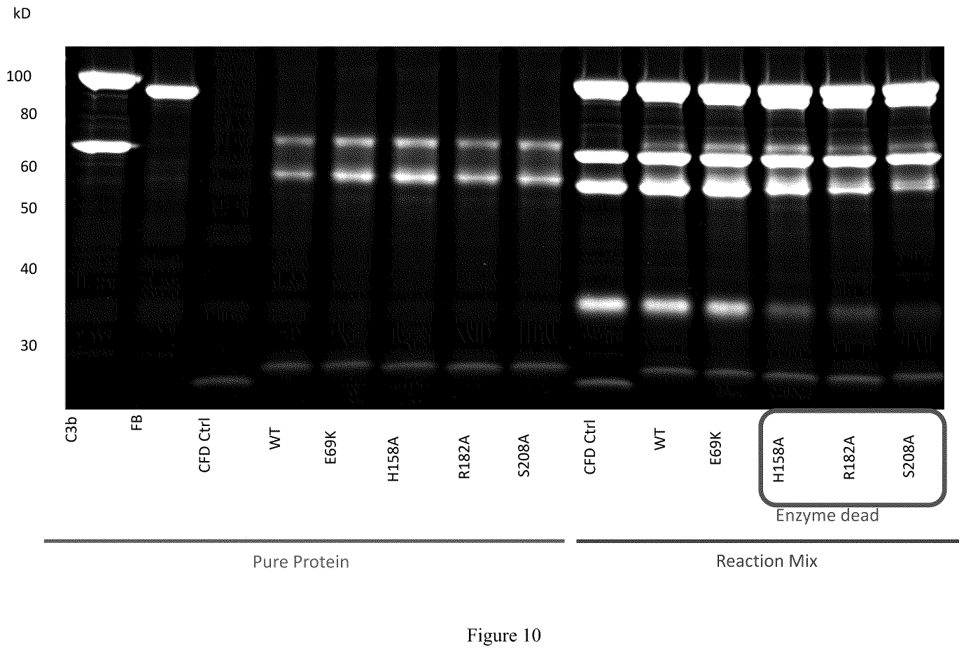

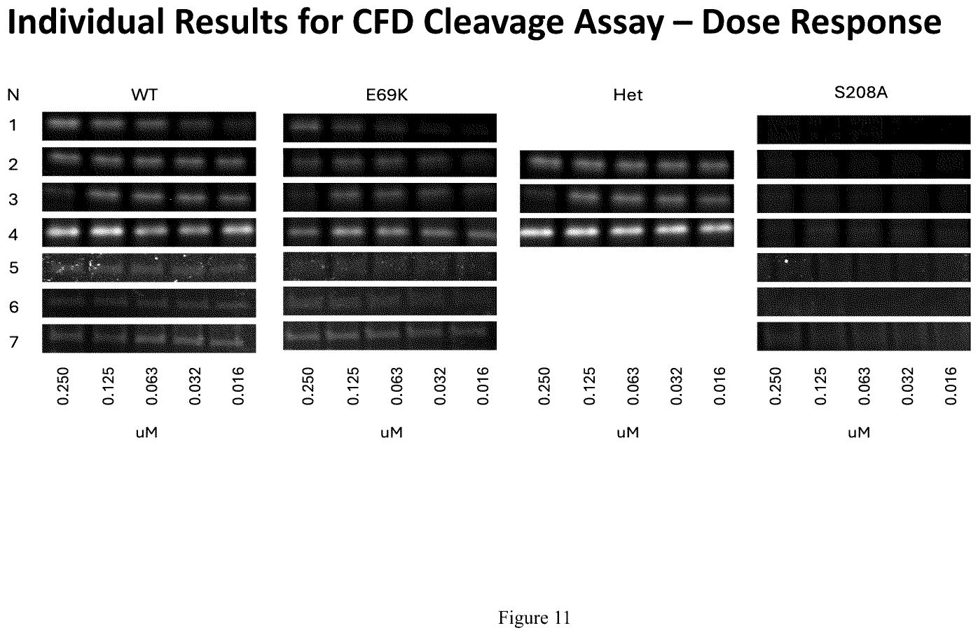

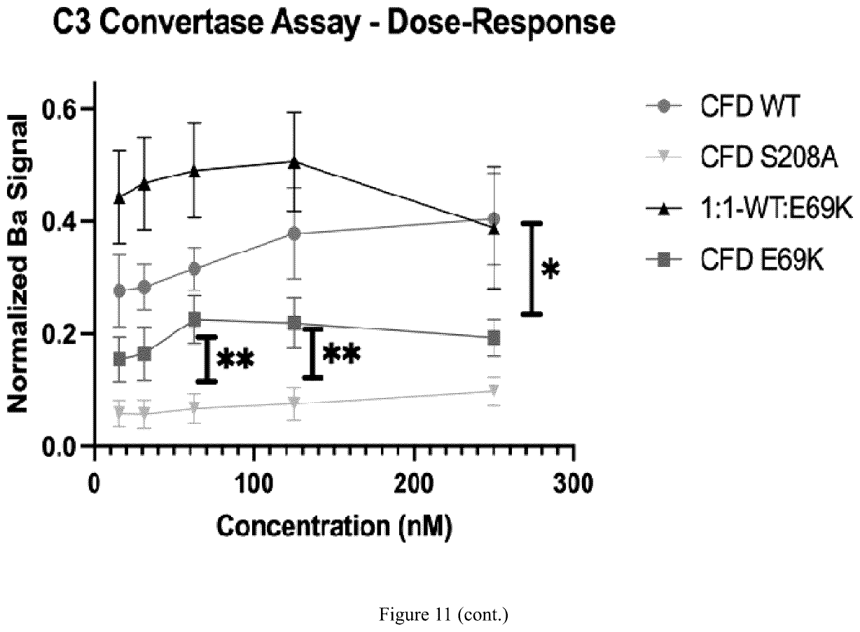

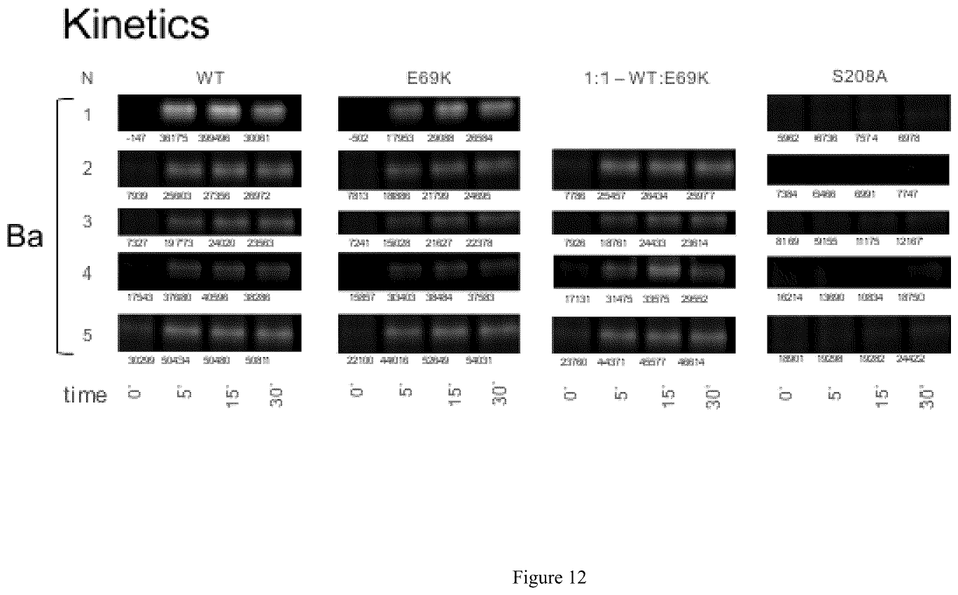

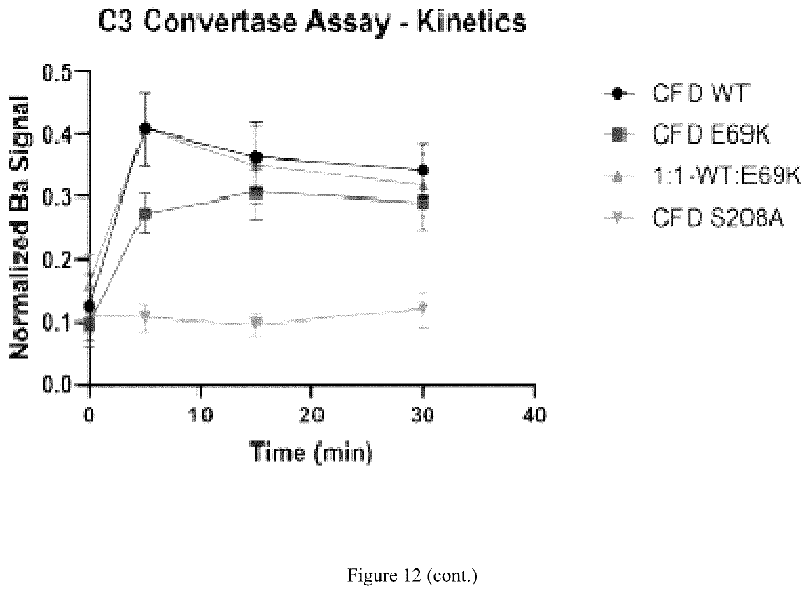

To understand the direction of effect on the protective CFD variant (E69K), a series of CFD-mutant recombinant proteins were produced and their catalytic activity was tested using an in vitro C3b/CFB convertase assay. Although the CFD E69K mutation was not within the active site, the factor B binding exosite, or the known inhibitory loop, this variant was found to reduce but does not completely abolish CFD catalytic activity. This identified a novel site of CFD function and suggested that even partial loss of function provides significant protection against AMD. In particular, CFD variants were produced and purified from transfected cells (see, FIG. 8). The C3b/CFB convertase assay uses purified proteins to test the function of CFD (see, FIG. 9). An assay validation is shown in FIG. 10. Dose-response and kinetics are shown in FIG. 11 and FIG. 12, respectively. FIG. 11 shows CFBa cleavage fragment band following an in vitro cleavage assay containing C3b, Fb, and the indicated CFD (WT, E69K mutant, or S208A mutant) at the doses indicated. FIG. 11 also shows in vitro cleavage assay containing C3b, CFB, and the indicated CFD (WT, E69K mutant, or S208A mutant) at indicated concentrations. CFBa cleavage fragment band intensity is quantified and normalized to C3b band intensity. Multiple unpaired t-tests, Sidak-Bonferroni corrected (*p<. 05, **p<0.005). The results indicated that: 1) produced CFD was functionally validated in C3b/CFB convertase assay, and 2) the CFD E69K variant demonstrated a significant reduction in alternative pathway activity. Together, the human genetics and this experimental data demonstrate that inhibitition of the AP function by targeting CFD may be sufficient to reduce overall complement activity.

The results from Examples 3-6 indicate: 1) CFD protein was mainly produced by adipose tissue, although CFD was also found in choroidal and RPE cells in the eye; 2) a CFD missense variant (19:860766:G:A, E69K) was identified to be protective against AMD; 3) the CFD E69K mutation was not in the active site, the factor B binding exosite, or the known inhibitory loop; 4) this mutation may stabilize the inactive conformation or interact negatively with the positioning of C3bB within the catalytic site for cleavage; and 5) this variant reduces but does not completely abolish CFD catalytic activity.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

What is claimed is:

1. A method of treating a subject having macular degeneration or at risk of developing macular degeneration, the method comprising administering a Complement Factor D (CFD) inhibitor to the subject.

2. The method of claim 1, wherein the macular degeneration is wet macular degeneration.

3. The method of claim 1, wherein the macular degeneration is dry macular degeneration.

4. The method of claim 1, wherein the macular degeneration is intermediate macular degeneration.

5. The method of any one of claims 1 to 4, wherein the CFD inhibitor comprises an inhibitory nucleic acid molecule that hybridizes to a CFD nucleic acid molecule.

6. The method of claim 5, wherein the inhibitory nucleic acid molecule comprises an antisense nucleic acid molecule, a small interfering RNA (siRNA), and/or a short hairpin RNA (shRNA).

7. The method of claim 6, wherein the inhibitory nucleic acid molecule comprises an ShRNA.

8. The method of claim 6, wherein the inhibitory nucleic acid molecule comprises an antisense nucleic acid molecule.

9. The method of any one of claims 1 to 8, wherein the subject is also administered a macular degeneration therapeutic agent or macular degeneration therapy.

10. The method of any one of claims 1 to 9, further comprising detecting the presence or absence of a CFD variant nucleic acid molecule in a biological sample from the subject.

11. The method of claim 10, further comprising administering a macular degeneration therapeutic agent in an amount that is the same as or less than a standard dosage amount or macular degeneration therapy to the subject when the CFD variant nucleic acid molecule is absent from the biological sample.

12. The method of claim 10, further comprising administering a macular degeneration therapeutic agent in an amount that is the same as or less than a standard dosage amount or macular degeneration therapy to the subject when the subject is heterozygous for the CFD variant nucleic acid molecule.

13. The method of any one of claims 10 to 12, wherein the CFD variant nucleic acid molecule comprises a splice-site variant, a stop-gain variant, a start-loss variant, a stop-loss variant, a frameshift variant, a missense variant, an in-frame indel variant, and/or a variant that encodes a truncated CFD variant polypeptide.

14. The method of any one of claims 10 to 13, wherein the CFD variant nucleic acid molecule comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:860714:C:A (Cys51Ter), 19:863142:CACCTCGGGCTCGCGCGT:C (Thr223LeufsTer115), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter).

15. A method of treating a subject having macular degeneration or at risk of developing macular degeneration by administering a macular degeneration therapeutic agent or macular degeneration therapy, the method comprising:
determining or having determined whether the subject has a Complement Factor D (CFD) variant nucleic acid molecule, by:
obtaining or having obtained a biological sample from the subject; and
performing or having performed a sequence analysis on the biological sample to determine if the subject has a genotype comprising a CFD variant nucleic acid molecule; and
administering or continuing to administer the macular degeneration therapeutic agent in an amount that is the same as or less than a standard dosage amount or macular degeneration therapy, and/or a CFD inhibitor to a subject that is CFD reference;
administering or continuing to administer the macular degeneration therapeutic agent in an amount that is the same as or less than a standard dosage amount or macular degeneration therapy, and/or a CFD inhibitor to a subject that is heterozygous for the CFD variant nucleic acid molecule; or
administering or continuing to administer the macular degeneration therapeutic agent in a standard dosage amount or macular degeneration therapy to a subject that is homozygous for the CFD variant nucleic acid molecule;
wherein the presence of the CFD variant nucleic acid molecule indicates the subject has a decreased risk of developing macular degeneration.

16. The method of claim 15, wherein the CFD inhibitor comprises an inhibitory nucleic acid molecule that hybridizes to a CFD nucleic acid molecule.

17. The method of claim 16, wherein the inhibitory nucleic acid molecule comprises an antisense nucleic acid molecule, a small interfering RNA (siRNA), and/or a short hairpin RNA (shRNA).

18. The method of claim 16, wherein the inhibitory nucleic acid molecule comprises an siRNA.

19. The method of claim 16, wherein the inhibitory nucleic acid molecule comprises an antisense nucleic acid molecule.

20. The method of any one of claims 15 to 19, wherein the subject is heterozygous for the CFD variant nucleic acid molecule, and the subject is administered or continued to be administered the macular degeneration therapeutic agent in an amount that is the same as or less than a standard dosage amount or macular degeneration therapy and the CFD inhibitor.

21. The method of any one of claims 15 to 19, wherein the subject is CFD reference, and the subject is administered or continued to be administered the macular degeneration therapeutic agent in an amount that is the same as or less than a standard dosage amount or macular degeneration therapy and the CFD inhibitor.

22. The method of any one of claims 15 to 21, wherein the CFD variant nucleic acid molecule comprises a splice-site variant, a stop-gain variant, a start-loss variant, a stop-loss variant, a frameshift variant, a missense variant, an in-frame indel variant, and/or a variant that encodes a truncated CFD variant polypeptide.

23. The method of any one of claims 15 to 22, wherein the CFD variant nucleic acid molecule comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:860714:C:A (Cys51Ter), 19:863142:CACCTCGGGCTCGCGCGT:C (Thr223LeufsTer115), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter).

24. A method of identifying a subject having an increased risk of developing macular degeneration, the method comprising:
determining or having determined the presence or absence of a Complement Factor D (CFD) variant nucleic acid molecule in a biological sample obtained from the subject;
wherein:
when the subject is CFD reference, then the subject has an increased risk of developing macular degeneration; and
when the subject is heterozygous or homozygous for the CFD variant nucleic acid molecule, then the subject has a decreased risk of developing macular degeneration.

25. The method of claim 24, wherein the CFD variant nucleic acid molecule is a splice-site variant, a stop-gain variant, a start-loss variant, a stop-loss variant, a frameshift variant, a missense variant, an in-frame indel variant, or a variant that encodes a truncated CFD variant polypeptide.

26. The method of claim 24 or claim 25, wherein the CFD variant nucleic acid molecule comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter).

27. The method of any one of claims 24 to 26, further comprising administering a macular degeneration therapeutic agent in an amount that is the same as or less than a standard dosage amount or macular degeneration therapy, and/or a CFD inhibitor to a subject that is CFD reference.

28. The method of claim 27, wherein the subject is CFD reference, and the subject is administered or continued to be administered the macular degeneration therapeutic agent in an amount that is the same as or less than a standard dosage amount or macular degeneration therapy and the CFD inhibitor.

29. The method of any one of claims 24 to 26, further comprising administering a macular degeneration therapeutic agent in an amount that is the same as or less than a standard dosage amount or macular degeneration therapy, and/or a CFD inhibitor to a subject that is heterozygous for a CFD variant nucleic acid molecule.

30. The method of claim 29, wherein the subject is heterozygous for the CFD variant nucleic acid molecule, and the subject is administered or continued to be administered the macular degeneration therapeutic agent in an amount that is the same as or less than a standard dosage amount or macular degeneration therapy and the CFD inhibitor.

31. The method of any one of claims 27 to 30, wherein the CFD inhibitor comprises an inhibitory nucleic acid molecule that hybridizes to a CFD nucleic acid molecule.

32. The method of claim 31, wherein the inhibitory nucleic acid molecule comprises an antisense nucleic acid molecule, a small interfering RNA (siRNA), and/or a short hairpin RNA (shRNA).

33. The method of claim 32, wherein the inhibitory nucleic acid molecule comprises an ShRNA.

34. The method of claim 32, wherein the inhibitory nucleic acid molecule comprises an antisense nucleic acid molecule.

35. A macular degeneration therapeutic agent for use in the treatment or prevention of macular degeneration in a subject having a Complement Factor D (CFD) variant nucleic acid molecule.

36. The macular degeneration therapeutic agent of claim 35, wherein the CFD variant nucleic acid molecule is a splice-site variant, a stop-gain variant, a start-loss variant, a stop-loss variant, a frameshift variant, a missense variant, an in-frame indel variant, or a variant that encodes a truncated CFD variant polypeptide.

37. The macular degeneration therapeutic agent of claim 35 or claim 36, wherein the CFD variant nucleic acid molecule comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:863142:CACCTCGGGCTCGCGCGT:C (Thr223LeufsTer115), 19:860714:C:A (Cys51Ter), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter).

38. A Complement Factor D (CFD) inhibitor for use in the treatment or prevention of macular degeneration in a subject that is CFD reference or is heterozygous for a CFD variant nucleic acid molecule.

39. The CFD inhibitor of claim 38, wherein the CFD variant nucleic acid molecule is a splice-site variant, a stop-gain variant, a start-loss variant, a stop-loss variant, a frameshift variant, a missense variant, an in-frame indel variant, or a variant that encodes a truncated CFD variant polypeptide.

40. The CFD inhibitor of claim 38 or claim 39, wherein the CFD variant nucleic acid molecule comprises the genetic variation rs35186399 (19:860766:G:A; Glu69Lys), 19:860741:G:A (Trp60Ter), 19:860775:T:G, 19:863105:GC:G (Pro211ArgfsTer101), 19:860933:C:A (Tyr95Ter), 19:861912:G:T (Glu191Ter), 19:860714:C:A (Cys51Ter), 19:863142:CACCTCGGGCTCGCGCGT:C (Thr223LeufsTer115), 19:861785:C:A (Cys148Ter), 19:861926:C:A (Cys195Ter), 19:860692:AG:A (Gln44HisfsTer2), 19:861826:GCCCGGACAGC:G (Pro163CysfsTer28), 19:860752:CG:C (Ala65ArgfsTer91), 19:859745:G:T (NA), or 19:860686:C:A (Ser42Ter).

41. The CFD inhibitor of any one of claims 38 to 40, wherein the CFD inhibitor comprises an inhibitory nucleic acid molecule that hybridizes to a CFD nucleic acid molecule.

42. The CFD inhibitor of claim 41, wherein the inhibitory nucleic acid molecule comprises an antisense nucleic acid molecule, a small interfering RNA (siRNA), and/or a short hairpin RNA (shRNA).

43. The CFD inhibitor of claim 42, wherein the inhibitory nucleic acid molecule comprises an siRNA.

44. The CFD inhibitor of claim 42, wherein the inhibitory nucleic acid molecule comprises an antisense nucleic acid molecule.
